(19) United States
(12) Patent Application Publication
(10) Pub. No.: US 2025/0236575 A1
(43) Pub. Date: Jul. 24, 2025

(54) DIRECT HYDROGENATION OF METAL CARBONATE AND RELATED SALTS TO METHANOL, METHANE AND METAL HYDROXIDE

(71) Applicant: UNIVERSITY OF SOUTHERN CALIFORNIA, Los Angeles, CA (US)

(72) Inventors: Alain GOEPPERT, Los Angeles, CA (US); Christopher J. KOCH, Los Angeles, CA (US); G.K. Surya PRAKASH, Los Angeles, CA (US); Raktim SEN, Los Angeles, CA (US)

(73) Assignee: UNIVERSITY OF SOUTHERN CALIFORNIA, Los Angeles, CA (US)

(21) Appl. No.: 18/703,290

(22) PCT Filed: Sep. 28, 2022
(86) PCT No.: PCT/US2022/045003

§ 371 Date: Apr. 19, 2024

Related U.S. Application Data
(60) Provisional application No. 63/270,540, filed on Oct. 21, 2021.

(51) Int. Cl. C07C 29/132; C07C 1/12

(52) U.S. Cl. CPC C07C 29/132; C07C 1/12; C07C 2531/12

(57) ABSTRACT

A liquid phase process for direct utilization of inorganic metal carbonate and related salts under hydrogenative conditions to produce value-added fuels and feedstocks such as methanol, methane, carbon monoxide, and higher hydrocarbons. Advantageously, the reaction can use heterogeneous catalysts. The hydrogenation proceeds with high selectivity and yield for the desired product at relatively low temperatures, along with co-production of metal hydroxide. The metal hydroxide can be used to capture CO₂, forming metal carbonates and bicarbonates that can be reused to produce more methanol and methane, hydrocarbons and carbon monoxide, closing the loop. Such a hydrogenation process can thus also be used in a “carbon capture and recycling” manner to produce renewable methane, methanol, carbon monoxide, hydrocarbons, and other carbon feedstocks from CO₂ sourced from any natural and anthropogenic emissions as well as from ambient air.

### CROSS-REFERENCE TO RELATED APPLICATIONS

This application claims the benefit of U.S. provisional application Ser. No. 63/270,540 filed Oct. 21, 2021, the disclosure of which is hereby incorporated in its entirety by reference herein.

### TECHNICAL FIELD

In at least one aspect, the present invention relates to the catalytic reduction of metal carbonate, bicarbonate, and alkyl carbonate salts using molecular hydrogen and heterogeneous catalysts to produce methanol, methane, carbon monoxide and other hydrocarbons (C_{≥2}) as well as metal hydroxide.

### BACKGROUND

In a sustainable and circular economy, renewable carbon feedstocks are of paramount importance. In this, the utilization of the CO₂ emitted from industrial exhausts such as flue gases of fossil fuel burning power plants, natural gas purification facilities, cement plants, biogas, biomass burning facilities, biomass gasification as well as directly from air (direct air capture; DAC) is an attractive “carbon capture and recycling/utilization (CCR/CCU)” technology. CO₂, from any CO₂ source including air, can be efficiently captured using metal hydroxide bases to form bicarbonate and carbonate salts. Existing technologies that utilize carbonate salts in particular, require high energy inputs (ΔGₘᵢₙ>100 KJ/mol) and temperatures (>800° C.) to desorb the CO₂ to its free gas form, and thereby allowing CO₂ to be utilized.

Metal carbonate salts are important carbon sources and are solid carriers of CO₂, which are convenient to store and transport. Various such salts are also relevant to daily life such as baking powder, baking soda (sodium bicarbonate), antacids (magnesium carbonate) and limestone (calcium carbonate). In the present invention, we disclose a low temperature alternate route to directly utilize carbonate/bicarbonate salts in the presence of a suitable solvent under reductive conditions using molecular H₂. The H₂ gas may be obtained from any available source through any known process. These processes include natural gas, methane and biogas reforming and partial oxidation (steam reforming, dry reforming, autothermal reforming and combinations thereof), coal gasification, petroleum reforming, biomass gasification, as well as water electrolysis and other water splitting methods using renewable energies such as wind, solar, geothermal or biomass. Depending on the choice of catalyst, the hydrogenation pathway can be altered to produce methanol and methane with high selectivity and yields.

Methanol (CH₃OH), also referred to as wood alcohol, is the simplest alcohol, a convenient one-carbon liquid at room temperature that is easy to store, transport and dispense. It is a prominent building block to synthesize various commodity chemicals and materials such as formaldehyde, methyl-tert-butyl ether (MTBE), acetic acid, dimethyl ether and various polymers, paints, adhesives, construction materials, pharmaceuticals and many others. Industrially, methanol is also catalytically converted to a variety of hydrocarbons and olefins such as ethylene, propylene, gasoline etc. through the methanol-to-olefin (MTO) and methanol-to-gasoline (MTG) processes. In essence, most of the carbon-based commodity chemicals and fuels presently obtained from fossil sources can be derived from methanol.

Besides its applications as a feedstock, methanol is an excellent fuel that is very clean burning. It is a direct drop-in fuel in internal combustion engines (ICE), gas turbines, cook stoves, boilers, and direct methanol fuel cells (DMFC). Furthermore, it is widely used as an additive to gasoline, notably due to its high-octane number. It can also be used in modified diesel engines with high efficiency producing high torque. The emissions from such engines are quite clean with very little production of soot and NOx. Methanol has also a tremendous hydrogen storage capacity (H₂ content=12.6 wt %), and the stored hydrogen can be easily extracted through methanol reforming and utilized in hydrogen fuel cells (reformed methanol fuel cell, RMFC). Hence, methanol is a suitable liquid organic hydrogen carrier (LOHC).

Similarly, methane which is the primary component of natural gas is a commonly used fuel and feedstock. Synthetic or substitute natural gas (SNG) is currently produced from fossil fuels or biofuels and is commercially used as fuel in the form of LNG or CNG (liquefied or compressed natural gas). When produced renewably, methane can be easily implemented as a sustainable energy medium delivered through the infrastructure that already exists for natural gas.

Additionally, carbon monoxide and its combination with H₂ (syngas) is an important feedstock to produce hydrocarbon-based chemicals, polymers and fuels on an industrial scale. Hence, it is desirable to develop a process for metal carbonate hydrogenation to access carbon monoxide as well as other hydrocarbons such as dimethyl ether (DME), ethane, ethylene, propylene, formic acid, and oxalic acid, among others, that are all industrially significant chemicals.

The transformation of metal carbonates and bicarbonates to carbon feedstocks and fuels such as methanol, methane, carbon monoxide, hydrocarbons and others required a least two steps: (1) the release of the CO₂ bound in the metal carbonates and bicarbonates at high temperatures and (2) the reduction of the released CO₂ to produce the desired product. What is described here is a process for the direct conversion of metal carbonates and bicarbonates to synthesize methane, methanol, carbon monoxide, hydrocarbons (C_{≥2}) and others.¹⁻³

Accordingly, there is a need for improved economical processes and methods for producing methanol, methane, carbon monoxide, hydrocarbons (C_{≥2}) or mixture thereof from CO₂ sources.

### SUMMARY

In at least one aspect, a method for conversion of a carbonate/formate component is provided. The method includes a step of contacting the carbonate/formate component with hydrogen to produce methanol, methane, carbon monoxide, hydrocarbons (C_{≥2}) or a mixture thereof over a catalyst in a solvent. Characteristically, the catalyst includes a transition metal, a post-transition metal, a lanthanide, or combinations thereof.

In another aspect, a method for the conversion of a metal carbonate, metal bicarbonate, metal formate salt, and mixtures thereof is provided. The method includes a step of contacting a metal carbonate, metal bicarbonate, metal formate salt, and mixtures thereof with hydrogen to produce methanol, methane, carbon monoxide, hydrocarbons (C_{≥2}), or a mixture thereof over a catalyst in a solvent. Characteristically, the catalyst including a transition metal, a post-transition metal, a lanthanide, or combinations thereof.

In another aspect, a method for producing methanol, methane, carbon monoxide, hydrocarbons (C_{≥2}), or a mixture thereof from CO₂ is provided. The method includes the following steps:
(a) capturing CO₂ with a metal hydroxide or metal hydroxide mixture from a CO₂ source containing between 0.04% and 100% CO₂, including ambient air, indoor air, industrial flue gases, fossil fuel burning power plants, natural gas purification facilities, cement plants, biogas, breweries, biomass burning facilities and biomass gasification facilities to obtain metal carbonates, metal bicarbonates or mixtures thereof;
(b) converting the metal carbonates, metal bicarbonates and mixtures thereof with hydrogen to produce methanol, methane, carbon monoxide, hydrocarbons, or a mixture thereof over a catalyst in a solvent while regenerating the metal hydroxide or metal hydroxide mixture; and
(c) reusing the metal hydroxide or metal hydroxide mixture to capture CO₂ as described in step (a).

In another aspect, the methods and processes set forth herein are run either under batch conditions, flow conditions, or continuous (flow) conditions.

In another aspect, a process that reduces costs, complexity, and energetic needs is provided. In addition, metal hydroxides are also generated in this process. These metal hydroxides can be used to capture CO₂ and form metal carbonates and bicarbonates that can be used again to produce more methanol, methane, carbon monoxide, hydrocarbons (C_{≥2}) and other products in a cyclic operation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIG. 1A. Examples of reactions of metal carbonates to methanol and methane.

FIG. 1B. Examples of reactions of metal bicarbonates to methanol and methane.

FIG. 2A. Reactions of metal carbonates to methanol and methane with recycling of the metal hydroxide to metal carbonate.

FIG. 2B. Reactions of metal bicarbonates to methanol and methane with recycling of the metal hydroxide to metal bicarbonate.

FIG. 3A. Reactions of metal carbonates and bicarbonates to carbon monoxide with recycling of the metal hydroxide to metal carbonate and/or bicarbonate.

FIG. 3B. Reactions of metal carbonates and bicarbonates to hydrocarbons (C_{≥2}) with recycling of the metal hydroxide to metal carbonate and/or bicarbonate.

FIG. 4. Closed cycle for the production of methanol, methane, carbon monoxide and other hydrocarbons from captured CO₂ and H₂ using metal hydroxide/carbonate/bicarbonate.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, alkylaryl (e.g., C₁₋₈ alkyl C₆₋₁₀ aryl), —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —OM+, —SO₃⁻M⁺, —PO₃⁻M⁺, —COO⁻M⁺, —CF₂H, —CF₂R′, —CFH₂, and —CFR′R″ where R′, R″ and R″ are C₁₋₁₀ alkyl or C₆₋₁₈ aryl groups, M⁺ is a metal ion, and L⁻ is a negatively charged counter ion; R groups on adjacent carbon atoms can be combined as —OCH₂O—; single letters (e.g., “n” or “o”) are 1, 2, 3, 4, or 5; in the compounds disclosed herein a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —OM+, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups, M⁺ is a metal ion, and L⁻ is a negatively charged counter ion; hydrogen atoms on adjacent carbon atoms can be substituted as —OCH₂O—; when a given chemical structure includes a substituent on a chemical moiety (e.g., on an aryl, alkyl, etc.) that substituent is imputed to a more general chemical structure encompassing the given structure; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B”. In the case of “only A”, the term also covers the possibility that B is absent, i.e. “only A, but not B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset. In a refinement, “one or more” includes “two or more.”

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1 to 2.1 the following numbers 1.2, 1.3, 1.4, 1.5, 1.6, 1.7, 1.8, 1.9, and 2.0 can be selected as lower or upper limits.

When referring to a numeral quantity, in a refinement, the term “less than” includes a lower non-included limit that is 5 percent of the number indicated after “less than.” For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, or 1 percent of the number indicated after “less than.”

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

For all compounds expressed as an empirical chemical formula with a plurality of letters and numeric subscripts (e.g., CH₂O), values of the subscripts can be plus or minus 50 percent of the values indicated rounded to or truncated to two significant figures. For example, if CH₂O is indicated, a compound of formula C_{(0.8-1.2)}H_{(1.6-2.4)}O_{(0.8-1.2)}. In a refinement, values of the subscripts can be plus or minus 30 percent of the values indicated rounded to or truncated to two significant figures. In still another refinement, values of the subscripts can be plus or minus 20 percent of the values indicated rounded to or truncated to two significant figures.

The term “alkali metal” means lithium, sodium, potassium, rubidium, cesium, and francium.

The “alkaline earth metal” means a chemical elements in group 2 of the periodic table. The alkaline earth metals include beryllium, magnesium, calcium, strontium, barium, and radium.

The term “transition metal” means an element whose atom has a partially filled d sub-shell, or which can give rise to cations with an incomplete d sub-shell. Examples of transition metals includes scandium, titanium, vanadium, chromium, manganese, iron, cobalt, nickel, copper, yttrium, zirconium, niobium, molybdenum, technetium, ruthenium, rhodium, palladium, silver, hafnium, tantalum, tungsten, rhenium, osmium, iridium, platinum, and gold.

The term “post-transition metal” means gallium, indium, tin, thallium, lead, bismuth. zinc, cadmium, mercury, aluminum, germanium, or antimony.

The term “lanthanide” or lanthanoid series of chemical elements” means an element with atomic numbers 57-71. The lanthanides metals includes lanthanum, cerium, praseodymium, samarium, europium, gadolinium neodymium, promethium, terbium, dysprosium, holmium, erbium, thulium, ytterbium, or lutetium.

The term “metal” as used herein means an alkali metal, an alkaline earth metal, a transition metal, a lanthanide, an actinide, or a post-transition metal.

The term “gauge pressure” as used herein means the pressure measured relative to the ambient atmospheric pressure.

Throughout this application, where publications are referenced, the disclosures of these publications in their entireties are hereby incorporated by reference into this application to more fully describe the state of the art to which this invention pertains.

### Abbreviations

“CCU” means carbon capture and utilization.

“CCS” means carbon capture and sequestration. CCR: carbon capture and recycling. Mmol: millimole.

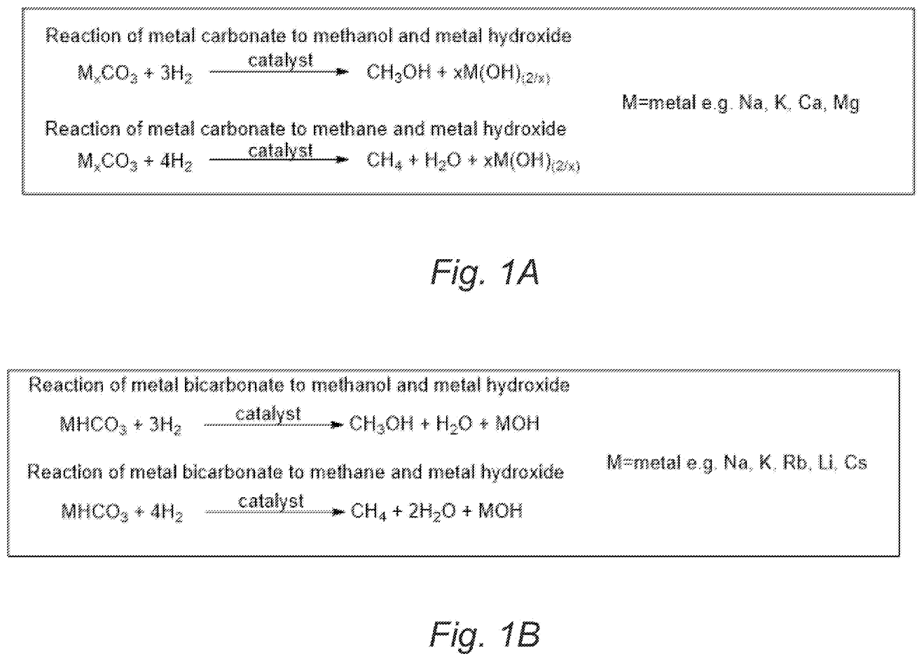

In at least one aspect, a liquid phase system for catalytic direct hydrogenation of metal carbonate and bicarbonate salts at moderate to low temperatures in presence of molecular H₂ is provided. Examples of these reactions of metal carbonates and bicarbonates to methanol and methane are provided in FIG. 1.

In another aspect, a method for the conversion of a carbonate/formate component is provided. The method includes a step of contacting the carbonate/formate component with hydrogen to produce methanol, methane, carbon monoxide, hydrocarbons, or a mixture thereof over a catalyst in a solvent. Characteristically, the catalyst includes a transition metal, a post-transition metal, a lanthanide, or combinations thereof. In a refinement, the carbonate/formate component is a metal carbonate, a metal bicarbonate, and/or a metal formate. In another refinement, the carbonate/formate component is an ammonium or tetraalkyl ammonium carbonate, an ammonium or tetraalkyl ammonium bicarbonate, and/or an ammonium or tetraalkyl ammonium formate. In a refinement, the alkyl groups in the tetraalkyl ammonium can be C₁₋₆ alkyl groups.

In another aspect, the reaction of the carbonate/formate component with hydrogen to produce methanol, methane, carbon monoxide, hydrocarbons, or a mixture thereof over a catalyst is performed under alkaline conditions. Therefore, the reaction is performed at a pH over 7. In some refinements, the reaction is performed at a pH greater than 7, 7.5, 8, 9, 10, 12 or 14. In a further refinement, the reaction is performed at a pH less than 14, 13, 12, or 10.

In another aspect, the reaction can be operated at ambient temperatures to high temperatures. The reaction efficiency and rates are higher at relatively moderate temperatures of 150° C. and above for methanol synthesis and the methanation reaction. For homogeneous catalysts, reaction temperature can be about 100° C. (e.g, 80 to 150° C.). The production of carbon monoxide, dimethyl ether, and mixtures of hydrocarbons is also possible. In a refinement, the reaction is performed at a temperature from about 20° C. to 250° C. In some variations, the reaction is performed at a temperature of at least 20° C., 30° C., 50° C., 70° C., 100° C., 120° C., 130° C., 140° C., or 120° C. In a refinement, the reaction is performed at a temperature of at most 700° C., 600° C., 500° C., 400° C., 350° C., 300° C., 350° C., 200° C., or 150° C.

In another aspect, the H₂ pressure can be varied from ˜1 atm to higher pressures depending on the activity of the catalyst and other reaction parameters. In the case of the reaction producing methane, it should be noted that the gaseous product adds to the pressure of the system unless released to operate at constant pressure. In some refinements, the reaction is conducted at gauge pressures of at least 0 bar, 1 bar, 5 bar, 10 bar, 50 bar, 80 bar, 100 bar, or 150 bar. In further refinement, the reaction is conducted at gauge pressures of at most 400 bar, 350 bar, 300 bar, 250 bar, 200 bar, 150 bar, 100 bar, or 50 bar. In one example, the conversion of the metal carbonate, metal bicarbonate, metal formate salt, and mixtures thereof with hydrogen to produce methanol, methane, carbon monoxide, hydrocarbons, or mixtures thereof is conducted at temperatures between 50° C. and 600° C. and gauge pressures between 0 and 300 bar.

In another aspect, high selectivities of 90% and above can be achieved for the desired product, while the product yields can also be in the higher range (>80%). In a refinement, very high selectivities of 95% and above can be achieved for the desired product, while the product yields are also in the higher range (>90%).

In another aspect, the hydrogen used in the reaction is produced from any fossil or renewable source including natural gas, petroleum, coal, biomass, biogas, and water. In another refinement, the hydrogen used in the reaction is produced electrochemically from water. In another refinement, the hydrogen used in the reaction is produced by any other method able to split water, including photochemical, thermal, and photovoltaic. In still another refinement, the hydrogen used in the reaction is produced electrochemically from water obtained as a side product of the carbonate/bicarbonate hydrogenation reaction.

In another aspect, the process can be operated under batch and flow conditions. The product separation is straightforward in both cases. Under flow conditions, the products can be collected from the outlet stream. Under batch conditions, methanol (b.p. 64.7° C.) can for example be distilled off from the reaction mixture. Methane gas or methane/hydrogen gas mixtures can be released from the reactor to a storage container.

In another aspect, a suitable solvent can be chosen for effective hydrogenation of the substrate and; the conditions depend on the desired product. In a refinement, the solvent includes water, an alcohol, a diol, a polyol or a mixture thereof. For methanol synthesis, solvent systems including polar protic solvents like water, alcohol, or a mixture of these solvents with a co-solvent may be used. Examples of alcohol solvents are methanol, ethanol, isopropanol, n-butanol, 2-methoxyethanol, ethylene glycol, diethylene glycol, triethylene glycol, polyethylene glycols, propylene glycol, 1,4-butanediol, 2,3-butanediol and various amino-alcohols. For methane production, water is a preferred solvent. In a refinement, the solvent includes water, methanol, ethanol, isopropanol, n-butanol, ethanol propanol, 2-methoxyethanol, ethylene glycol, diethylene glycol, triethylene glycol, tetraethylene glycol, polyethylene glycol, propylene glycol, 1,4-butanediol or a mixture thereof.

In another aspect, a heterogeneous catalyst system includes the catalyst to produce methanol, methane, carbon monoxide hydrocarbons, or a mixture thereof set forth above. In a refinement, the heterogeneous catalyst system includes a component selected from the group consisting of copper-based catalysts, indium-based catalysts, nickel-based, indium and nickel/gallium-based catalysts modified with lanthanides and/or precious metals, and combinations thereof. In a refinement, the heterogeneous catalyst system includes an indium-based having formula In₂O₃/ZrO₂. In another refinement, the heterogeneous catalyst system includes a nickel-based catalyst selected from the group consisting of NiGa/SiO₂, Ni₃Ga/SiO₂, and Ni₅Ga₃/SiO₂. In a further refinement, the heterogeneous catalyst system further includes additional components selected from the group consisting of additives, support, and combinations thereof. Examples for the additional components include but are not limited to ZnO, ZrO₂, MOFs, Ga, Al₂O₃, SiO₂. TiO₂, or combinations thereof.

In another aspect, heterogeneous catalysts for carbonate, bicarbonate, and formate hydrogenation to methanol may include (but are not limited to) copper-based catalysts (with additives and supports such as ZnO, ZrO₂, MOFs, Ga, Al₂O₃, SiO₂. TiO₂), indium based (In₂O₃/ZrO₂), nickel based (NiGa/SiO₂, Ni₃Ga/SiO₂, Ni₅Ga₃/SiO₂), ruthenium, platinum, palladium based (Ru, Pt, Pd on various supports including silica, alumina, titania, zirconia, silica-alumina and carbon). Various promoters, such as precious metals (such as Pd, Pt, Rh, Ru) or lanthanides (such as La, Sm, Gd), can also be added to these heterogenous catalysts to improve selectivity and reactivity.

In another aspect, for methane and carbon monoxide synthesis, heterogeneous catalysts may include (but are not limited to) ruthenium, rhodium, nickel, cobalt, platinum, palladium, iron, iridium, etc. on a support (e.g., silica, alumina, titania, ceria, zirconia, silica-alumina etc.). Additives and promoters can be added to improve conversion and selectivities. In addition, homogeneous catalysts can also be used for this reaction. In a refinement, the catalyst to produce methane and carbon monoxide includes a heterogeneous catalyst based on nickel, cobalt, ruthenium, platinum, palladium, iron, iridium or rhodium and combinations thereof either in their pure form or deposited on a support including silica, alumina, zirconia, titanium oxide, cerium oxide and silica-alumina or mixtures thereof.

In another aspect, the heterogeneous catalyst are shaped into various forms that may include (but not limited to) granules, pellets, rings, tablets, spheres, cylinders and hollow cylinders.

In another aspect, the heterogeneous catalyst may be a powder.

In another aspect, the catalysts used herein have an average particle size less than or equal to 250 μm. In some variations, the catalysts used herein have an average particle size greater than or equal to 50 nm, 100 nm, 500 nm, 1 μm, 5 μm, 10 μm, 50 μm, or 100 μm. In some refinements, the catalysts used herein have an average particle size less than or equal to or 300 μm, 250 μm, 200 μm, 150 μm, 100 μm, 50 μm, 10 μm, 1 μm, or 500 nm.

In another aspect, the catalyst to produce methane and carbon monoxide by hydrogenation of metal carbonates, metal bicarbonates, and metal formate includes a homogenous catalyst based on a metal including (but not limited to) ruthenium, rhodium, nickel, cobalt, platinum, palladium, iron, iridium, and an organic ligand such as, but not limited to, pincer-type ligands. As used herein, when a catalyst is referred to as being based on a specified metal, it means that the catalyst includes the specified metal in an appropriate oxidation state to function as a catalyst for the reaction.

In another aspect, the catalyst to produce methanol by hydrogenation of metal carbonate, bicarbonate and formate includes a homogenous catalyst based on a metal including (but not limited to) ruthenium, rhodium, nickel, manganese, cobalt, platinum, palladium, iron, iridium, and an organic ligand, such as, but not limited to, pincer-type ligands.

In another aspect, for the production of mixtures of hydrocarbons, a Fischer-Tropsch type catalyst can be employed. These include typical Fischer-Tropsch type catalysts based on iron, cobalt, and ruthenium. In a refinement, the catalyst to produce a mixture of hydrocarbons comprises catalysts based on iron, copper, molybdenum, cobalt, ruthenium, metal carbides, zeolites or mixtures thereof.

In another aspect, for the production of other hydrocarbons such as dimethyl ethers, olefins, higher carbon alcohols, liquid hydrocarbons; and their mixtures, any metal catalyst based on prior art of transforming CO₂ gas into such chemicals can be employed. These may include catalysts containing iron, copper, molybdenum, cobalt, metal carbides, zeolites, etc., and mixtures thereof.

In another aspect, apart from a single class of catalysts, various combinations of such catalysts can be used to obtain the desired hydrocarbon products.

In another aspect, the metal carbonate and metal bicarbonate are products of a reaction of a metal hydroxide with CO₂ contained from various sources such as fossil fuel power plant flue gases, emissions from industrial and commercial sources and CO₂ contained in air.

In another aspect, the metal carbonate and metal bicarbonate are products of a reaction of a metal phosphate with CO₂ contained in various sources such as fossil fuel power plant flue gases, emissions from industrial and commercial sources and CO₂ contained in air. Examples of metal phosphates include tripotassium phosphate (K₃PO₄), dipotassium phosphate (K₂HPO₄), trisodium phosphate (Na₃PO₄), disodium phosphate (Na₂HPO₄), trilithium phosphate (Li₃PO₄) and dilithium phosphate (Li₂HPO₄).

In another aspect, the metal carbonate, metal bicarbonate, metal formate salt, and mixtures thereof react to form a metal hydroxide or metal hydroxide mixture.

In another aspect, the substrates include carbonate, bicarbonate/hydrogen carbonate, alkyl carbonate and formate salts of any metal including alkali metal, alkali earth metal, transition metal as well as rare earth metal. Examples of metal carbonates include sodium carbonate, potassium carbonate, calcium carbonate, magnesium carbonate, barium carbonate, cesium carbonate, lithium carbonate, rubidium carbonate, cesium carbonate, strontium carbonate, barium carbonate or mixtures thereof. Examples of metal bicarbonate include lithium bicarbonate, sodium bicarbonate, potassium bicarbonate, rubidium bicarbonate, cesium bicarbonate or mixtures thereof. Examples of metal formate salt include lithium formate, sodium formate, potassium formate, rubidium formate, cesium formate, or mixtures thereof.

In another aspect, the carbonate and bicarbonate salts may be obtained from any source. For example, commercially available metal carbonate and bicarbonate salts can be used. The carbonate/formate component is a metal carbonate and/or a metal bicarbonate each independently including a component selected from naturally occurring minerals; seashells, oyster shells, eggshells; carbonates and bicarbonate salts made by direct CO₂ capture from CO₂ sources (e.g, point source or air (DAC)) using alkali hydroxides and/or amines; synthetic (i.e., man-made) carbonates and bicarbonates; and combinations thereof. In a refinement, the carbonate/formate component includes naturally occurring minerals are selected from the group consisting of calcite (CaCO₃), magnesite (MgCO₃), siderite (FeCO₃), aragonite (CaCO₃), witherite (BaCO₃), natrite (Na₂CO₃), ankerite (CaFe—CO₃), dolomite (CaMg(CO₃)₂), huntite (Mg₃Ca(CO₃)₄), minrecordite (CaZn(CO₃)₂), barytocalcite (BaCa(CO₃)₂), hydromagnesite (Mg₅(CO₃)₄(OH)₂·4H₂O), ikaite (CaCO₃·6(H₂O)), lansfordite (MgCO₃·5(H₂O)), monohydrocalcite (CaCO₃·H₂O), natron (Na₂CO₃·10(H₂O)), and combinations thereof. In some variations, “carbon capture and recycling” technologies can be deployed. For example, CO₂ can be captured from any source including industrial and commercial flue gases, breweries, exhaust streams from various sources including transportation and indoor or ambient air on any metal hydroxide either in solid form or in a solvent to form the corresponding metal carbonate/bicarbonate salts. The capture can be performed at temperatures from −20° C. and up to the decomposition temperature of the corresponding carbonate salt, and an inlet gas stream pressure of about 1 atm or higher. The inlet stream for capture may be dry or humid. The obtained carbonate/bicarbonate salts can be processed further for hydrogenation.

Parallel to the carbon-based product (methane, methanol, carbon monoxide, or hydrocarbon mixtures or mixtures thereof), co-production of metal hydroxide or hydrated oxides can be achieved during the reaction.

Advantageously, such processes described herein based on carbonate/bicarbonate reduction can be developed as an energy and cost-effective process to produce metal hydroxides from their respective carbonate or bicarbonate feedstock in an alternate to the common thermal decomposition processes at very high temperatures utilizing a multi-step process.^{1,4}

The co-production of metal hydroxide in the described reaction offers a way to produce methanol, methane, carbon monoxide, hydrocarbons and derived products from any CO₂ source available. The recycling of the metal hydroxide at the end of the reaction to capture CO₂ and form more carbonate and bicarbonate allows for the metal hydroxide to be reused over many hydrogenation cycles with no or minimal need for the input of fresh metal hydroxide.

Furthermore, the convenient capture of CO₂ from a sustainable and renewable source such as biomass, biogas, breweries, and ambient air and reaction of the formed metal carbonate and bicarbonate with renewable hydrogen allows for the production of renewable methanol, methane and hydrocarbons. These products and their derivatives have a low or neutral carbon footprint because the CO₂ released by their utilization was captured from a renewable source. The capture, utilization and recycling of renewable CO₂ with hydrogen generated using renewable electrical energy, essentially constitutes a sustainable anthropogenic version of nature's own carbon cycle.

In another aspect, a method to produce methanol, methane, carbon monoxide, hydrocarbons or a mixture thereof from CO₂ is provided. The method includes the following steps:
(a) capturing CO₂ with a metal hydroxide or metal hydroxide mixture from a CO₂ source containing between 0.04% and 100% CO₂ to obtain metal carbonates, metal bicarbonates or mixtures thereof;
(b) converting the metal carbonates, metal bicarbonates and mixtures thereof with hydrogen to produce methanol, methane, carbon monoxide, hydrocarbons or a mixture thereof over a catalyst in a solvent while regenerating the metal hydroxide or metal hydroxide mixture; and
(c) reusing the metal hydroxide or metal hydroxide mixture to capture CO₂ as described in step (a).

Examples of suitable CO₂ sources include ambient air, indoor air, industrial flue gases, fossil fuel burning power plants, natural gas purification facilities, cement plants, biogas, breweries, biomass burning facilities and biomass gasification facilities

A particular embodiment of the invention disclosed herein is the capture of CO₂ from air (direct air capture, DAC) using any available technology with a metal hydroxide to form metal carbonate, metal bicarbonate or mixtures thereof and further reacting these metal carbonate/bicarbonate with hydrogen in the presence of a catalyst in a liquid phase to make products including but not limited to methanol, methane, and hydrocarbons. At the same time, during the hydrogenation reaction, the metal hydroxide is regenerated and can be reused to capture more CO₂ from the air. This allows the cycle to be closed on the metal hydroxide/carbonate/bicarbonate. Furthermore, once the hydrogenation products are used, all or part of the generated CO₂ will be released back the atmosphere. This allows for the carbon cycle to be closed as well.

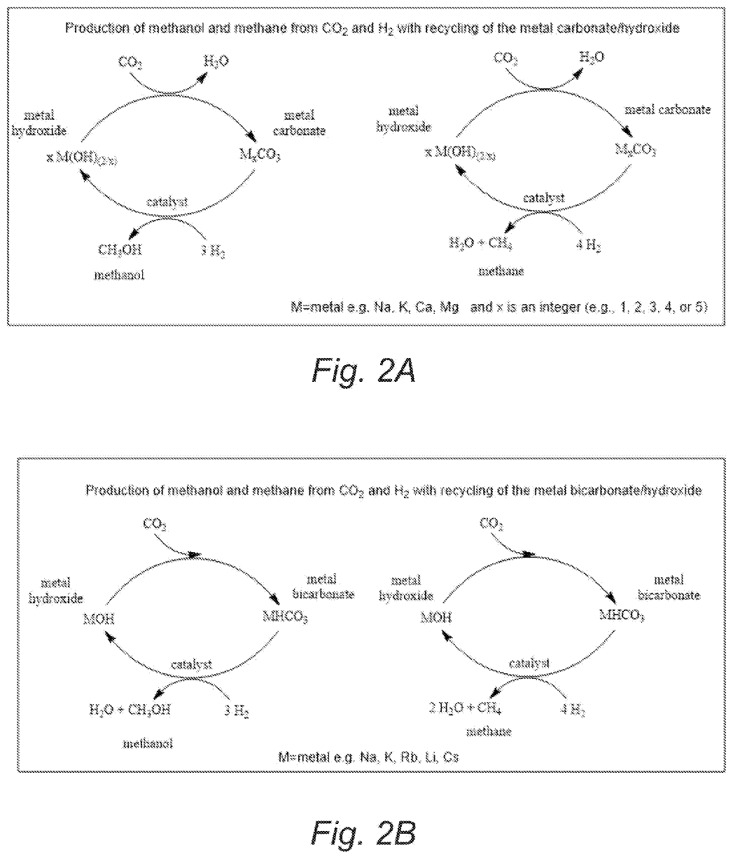

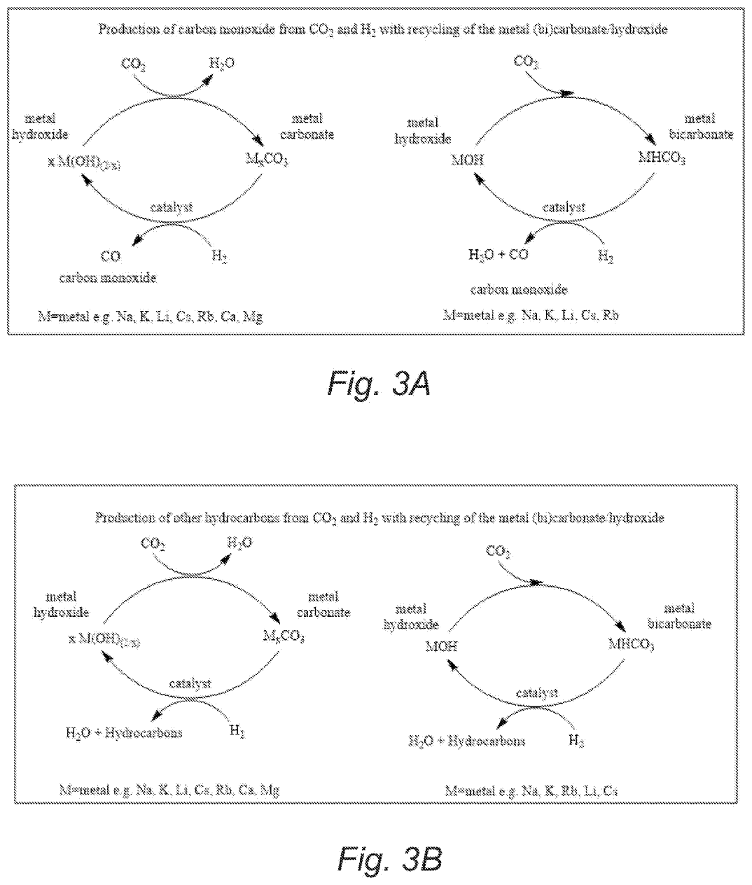

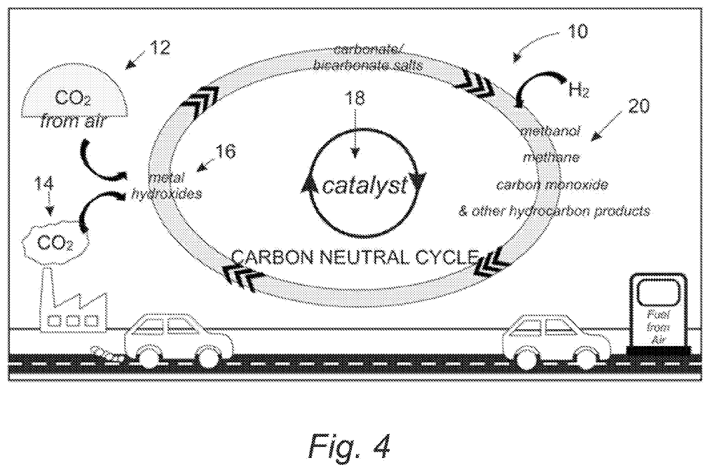

Illustrative examples of closed cycles for the production of methanol, methane, carbon monoxide and other hydrocarbons (C_{≥2}) from captured CO₂ and H₂ using metal hydroxide/carbonate/bicarbonate are given in FIGS. 2A, 2B, 3A and 3B as well as FIG. 4. FIG. 4 provides a schematic of a closed cycle for the production of methanol, methane, carbon monoxide and other hydrocarbons from captured CO₂ and H₂ using metal hydroxide/carbonate/bicarbonate. In this figure closed cycle 10 obtained CO₂ from ambient air 12 and/or industrial plant 14. The captured CO₂ is reacted with metal hydroxides 16 and then catalyst system 18 and molecular hydrogen as set forth above. The resulting products 20 include methanol, methane, carbon monoxide, and other hydrocarbon products. Advantageously, the metal carbonates and/or the metal bicarbonates and/or the metal hydroxides can be recycled and reused.

In still another aspect, the metal hydroxide includes lithium hydroxide, sodium hydroxide, potassium hydroxide, rubidium hydroxide, cesium hydroxide, magnesium hydroxide, calcium hydroxide, strontium hydroxide, barium hydroxide or mixtures thereof.

In a further aspect, the metal hydroxide produced can be used for ocean alkalinity enhancement (also called ocean alkalinization) by forming metal carbonate and bicarbonates with dissolved CO₂ when added to seawater.

In yet a further aspect, the products obtained by the hydrogenation of metal carbonates and bicarbonates (methane, methanol, carbon monoxide, hydrocarbons (C_{≥2}) and others) can be combusted in a stream of oxygen or air and the resulting CO₂ captured and sequestered or reused (CCS or CCU).

In still another aspect the hydrogen for the hydrogenation reaction is produced electrochemically from water obtained as a side product of the carbonate/bicarbonate hydrogenation reaction or from water obtained as a side product of CO₂ capture from various sources including capture of CO₂ from the air.

In still another aspect the hydrogen for the hydrogenation reaction is produced electrochemically from an aqueous solution of carbonate/bicarbonate used subsequently for the carbonate/bicarbonate hydrogenation reaction.

In still another aspect the hydrogen for the hydrogenation reaction is produced electrochemically from an aqueous solution of metal hydroxide obtained as a product reaction of the carbonate/bicarbonate hydrogenation reaction.

In yet another aspect, the hydrogen (H₂) for the hydrogenation reaction is produced in-situ, inside the hydrogenation reactor by water electrolysis.

In an additional aspect hydrogen can be generated from other sources including dehydrogenation of a variety of alcohols or another process known to those skilled in the art for producing hydrogen.

In a further aspect a method for producing methanol, methane, carbon monoxide, hydrocarbons or a mixture thereof from CO₂ is provided. The method includes the following steps:
(a) capturing CO₂ with a metal phosphate or metal phosphate mixture from a CO₂ source containing between 0.04% and 100% CO₂ to obtain metal carbonates, metal bicarbonates or mixtures thereof;
(b) converting the metal carbonates, metal bicarbonates and mixtures thereof with hydrogen to produce methanol, methane, carbon monoxide, hydrocarbons, or a mixture thereof over a catalyst in a solvent while regenerating the metal phosphate or metal phosphate mixture; and
(c) reusing the metal phosphate or metal phosphate mixture to capture CO₂ as described in step (a).

The following examples illustrate various embodiments of the present invention. Those skilled in the art will recognize many variations that are within the spirit of the present invention and scope of the claims.

### Example 1. Hydrogenation of Potassium Carbonate to Methanol Using Cu/ZnO/Al₂O₃Under Various Conditions

Commercial Cu/ZnO/Al₂O₃ catalyst was purchased from Alfa Aesar (63.5 wt % CuO, 24.7 wt % ZnO, 10.1 wt % Al₂O₃, 1.3 wt % MgO fume). The pellets were then crushed and sieved to a size of 250 μm or less. For activating the Cu/ZnO/Al₂O₃ catalyst, the catalyst was subjected to a flow of N₂ (100 mL/min) at 120° C. for 1 h, then the temperature was ramped up to 270° C. (at 10° C./min) with an H₂ flow (35 mL/min) in N₂ (100 mL/min) at 1 atm and kept at that temperature for 5 h. The so-treated catalyst was then stored under Argon for use at a later time. In a nitrogen-filled chamber, potassium carbonate, pre-activated catalyst, and the solvent were introduced to a 125 mL Parr reactor. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. Afterwards, the gases inside the vessel were partly collected in a gas sampling bag for GC analysis whereas the remaining gas was slowly released. The methanol formed was detected by ¹H NMR and isolated in vacuo. The results of the hydrogenation of potassium carbonate to methanol following the method described here are shown in Table 1.

TABLE 1
Hydrogenation of potassium carbonate to methanol
with Cu/ZnO/Al₂O₃ under various conditions:

| | | | Cu/ZnO/ | | | |
|---|---|---|---|---|---|---|
| | Temper- | | Al₂O₃ | | Product | |
| | ature | Pressure | Loading | Time | Methanol | Methanol |
| Trial | (° C.) | (bar) | (mg) | (hours) | (mmol) | Yield (%) |
| 1 | 170 | 70 | 300 | 48 | 0.7 | 7 |
| 2 | 200 | 70 | 300 | 48 | 5.6 | 56 |
| 3 | 250 | 70 | 300 | 48 | 7.2 | 72 |
| 4 | 200 | 60 | 300 | 48 | 5.4 | 54 |
| 5 | 200 | 80 | 300 | 48 | 5.8 | 58 |
| 6 | 200 | 70 | 150 | 48 | 1.9 | 19 |
| 7 | 200 | 70 | 450 | 48 | 7.4 | 74 |
| 8 | 200 | 70 | 300 | 24 | 4.6 | 46 |
| 9 | 200 | 70 | 300 | 72 | 7.1 | 71 |
| 10 ^{[a]} | 200 | 70 | 300 | 72 | 9.9 | 99 |

Reaction conditions: 10 mmol potassium carbonate, pressure of H₂ at room temperature, 800 rpm magnetic stirring, 10 mL of ethylene glycol.
^{[a]} 5 mL of ethylene glycol, no magnetic stirring. CH₃OH yields calculated relative to the carbonate as determined by ¹H NMR. Yield calculations ±5%.

### Example 2. Hydrogenation of Some Metal Carbonates to Methanol

Commercial Cu/ZnO/Al₂O₃ catalyst was purchased from Alfa Aesar (63.5 wt % CuO, 24.7 wt % ZnO, 10.1 wt % Al₂O₃, 1.3 wt % MgO fume). The pellets were then crushed and sieved to a size of 250 μm or less. For activating the Cu/ZnO/Al₂O₃ catalyst, the catalyst was subjected to a flow of N₂ (100 mL/min) at 120° C. for 1 h, then the temperature was ramped up to 270° C. (at 10° C./min) with an H₂ flow (35 mL/min) in N₂ (100 mL/min) at 1 atm and kept at that temperature for 5 h. The so-treated catalyst was then stored under Argon for use at a later time. In a nitrogen-filled chamber, the substrate, pre-activated catalyst, and the solvent were introduced to a 125 mL Parr reactor. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. Afterwards, the gases inside the vessel were partly collected in a gas sampling bag for GC analysis whereas the remaining gas was slowly released. The methanol formed was detected by ¹H NMR and isolated in vacuo. The results of the hydrogenation of some metal carbonates to methanol following the method described here are shown in Table 2.

TABLE 2
Hydrogenation of some metal carbonates to methanol

| | Products | | |
|---|---|---|---|
| Carbonate | Formate | Methanol | Methanol |
| used | (mmol) | (mmol) | Yield (%) |
| Li₂CO₃ | 0 | 1.1 | 11 |
| Na₂CO₃ | 0 | 5.9 | 59 |
| K₂CO₃ | 0.1 | 9.9 | 99 |
| Rb₂CO₃ | 0.9 | 8.7 | 87 |
| Cs₂CO₃ | 0.9 | 6.2 | 62 |
| MgCO₃ | 0 | 1.2 | 12 |
| CaCO₃ | 0 | 1.2 | 12 |
| BaCO₃ | 0 | 0.9 | 9 |
| MgCO₃^{[a]} | 0 | 4.8 | 48 |
| CaCO₃^{[a]} | 0 | 6.0 | 60 |
| BaCO₃^{[a]} | 0 | 4.6 | 46 |

Reaction conditions: 10 mmol carbonate salt, ethylene glycol (5 mL), 70 bar H₂ at room temperature, 200° C., 72 hours, 300 mg Cu/ZnO/Al₂O₃.
^{[a]}85 bar H₂ at room temperature, 250° C., 800 rpm magnetic stirring. CH₃OH yields calculated relative to the carbonate as determined by ¹H NMR. Yield calculations ±5%.

### Example 3. Hydrogenation of Some Metal Bicarbonates and Formates to Methanol

Commercial Cu/ZnO/Al₂O₃ catalyst was purchased from Alfa Aesar (63.5 wt % CuO, 24.7 wt % ZnO, 10.1 wt % Al₂O₃, 1.3 wt % MgO fume). The pellets were then crushed and sieved to a size of 250 μm or less. For activating the Cu/ZnO/Al₂O₃ catalyst, the catalyst was subjected to a flow of N₂ (100 mL/min) at 120° C. for 1 h, then ramped up to 270° C. (at 10° C./min) with a H₂ flow (35 mL/min) in N₂ (100 mL/min) at 1 atm and kept at that temperature for 5 h. The so treated catalyst was then stored afterwards under Argon for use at a later time. In a nitrogen-filled chamber, the substrate, pre-activated catalyst and the solvent were introduced to a 125 mL Parr reactor with a magnetic stirrer. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. Afterwards, the gases inside the vessel were partly collected in a gas sampling bag for GC analysis whereas the remaining gas was slowly released. The methanol formed was detected by ¹H NMR and isolated in vacuo. The results of the hydrogenation of some metal carbonates and metal formates to methanol following the method described here are shown in Table 3.

TABLE 3
Hydrogenation of some metal bicarbonates
and formates to methanol

| | Products | | |
|---|---|---|---|
| | Formate | Methanol | Methanol |
| Substrate | (mmol) | (mmol) | yield (%) |
| NaHCO₃ | 0 | 6.2 | 62 |
| NaHCO₂ | 0 | 7.1 | 71 |
| KHCO₃ | 0 | 9.7 | 97 |
| KHCO₂ | 0 | 9.8 | 98 |
| CsHCO₃ | 1.3 | 8.7 | 87 |
| CsHCO₂ | 0 | 8.2 | 82 |
| LiHCO₂ | 0 | 1.3 | 13 |
| Mg(HCO₂)₂^{[a]} | 0 | 2.8 | 28 |
| Ca(HCO₂)₂^{[a]} | 0 | 0.8 | 8 |

Reaction conditions: 10 mmol salt(substrate), ethylene glycol (5 mL), 200° C., 70 bar H₂, 72 hours, 300 mg Cu/ZnO/Al₂O₃
^{[a]}Reaction conditions: 5 mmol salt, ethylene glycol (5 mL), 200° C., 70 bar H₂, 72 hours, 300 mg Cu/ZnO/Al₂O₃. CH₃OH yields calculated relative to the carbonate as determined by ¹H NMR. Yield calculations ±5%.

### Example 4. Hydrogenation of Carbonates in Biomass Samples

Commercial Cu/ZnO/Al₂O₃ catalyst was purchased from Alfa Aesar (63.5 wt % CuO, 24.7 wt % ZnO, 10.1 wt % Al₂O₃, 1.3 wt % MgO fume). The pellets were then crushed and sieved to a size of 250 μm or less. For activating the Cu/ZnO/Al₂O₃ catalyst, the catalyst was subjected to a flow of N₂ (100 mL/min) at 120° C. for 1 h, then the temperature was ramped up to 270° C. (10° C./min) with a H₂ flow (35 mL/min) in N₂ (100 mL/min) at 1 atm and kept at that temperature for 5 h. The so treated catalyst was then stored under Argon for use at a later time. The biomaterial was crushed and sieved to a fine powder form. In a nitrogen-filled chamber, the biomaterial substrate, pre-activated catalyst and the solvent were introduced to a 125 mL Parr reactor with a magnetic stirrer. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. Afterwards, the gases inside the vessel were partly collected in a gas sampling bag for GC analysis whereas the remaining gas was slowly released. The methanol formed can be detected by ¹H NMR and isolated in vacuo. The results of the hydrogenation of some bio-materials to methanol following the method described here are shown in Table 4.

TABLE 4
Hydrogenation of carbonates in biomass samples

| Starting material | | | Products | | |
|---|---|---|---|---|---|
| Bio- | Amount | % | Formate | Methanol | Methanol |
| material | (g) | CaCO₃ | (mmol) | (mmol) | Yield (%) |
| Seashell | 1.0 | 99.6% | traces | 5.8 | 58 |
| Eggshell | 1.0 | 57.3% | traces | 3.2 | 56 |

Reaction conditions: 1 gram bio-material, ethylene glycol (10 mL), 85 bar H₂ at room temperature, 250° C., 72 hours, 800 rpm, and 300 mg Cu/ZnO/Al₂O₃. CH₃OH yields calculated relative to the carbonate as determined by ¹H NMR. Yield calculations ±5%.

### Example 5. CO₂ Capture and Hydrogenation to Methanol

CO₂ capture from pure CO₂ stream: A known amount of alkali hydroxide (KOH) was dissolved in ethylene glycol (10 mL) in a vial with a magnetic stir bar. The gases inside the vial were then removed under vacuum. CO₂ was subsequently added while stirring the solution at 800 rpm for 3 h and maintaining the CO₂ pressure inside the reactor at about 1 psi above atmospheric pressure. The amounts of CO₂ captured were calculated through gravimetric analysis of the solutions before and after the capture.

CO₂ capture from air: In a 30 mL vial, a specific amount of KOH was dissolved in 10 mL ethylene glycol. Atmospheric air containing ˜420 ppm CO₂ was then bubbled through the solution at a flowrate of 200 mL/min for 72 h using a pump. The resulting solution was then sparged with N₂ for 2 h. Afterwards, imidazole (Im) was added as an internal standard to 0.5 mL aliquot of the homogeneous solution that was analyzed by ¹H and ¹³C NMR with DMSO-d₆ as the deuterated solvent. The amount of CO₂ captured was calculated through ¹³C NMR analysis. The remaining solution was used for hydrogenation.

Hydrogenation of captured carbonate species: For activating the Cu/ZnO/Al₂O₃ catalyst, the catalyst was subjected to a flow of N₂ (100 mL/min) at 120° C. for 1 h. Then the temperature was ramped up to 270° C. (at 10° C./min) with a H₂ flow (35 mL/min) in N₂ (100 mL/min) at 1 atm and kept at that temperature for 5 h. The so treated catalyst was then stored under Argon for use at a later time. In a nitrogen-filled chamber, the CO₂ loaded ethylene glycol solution and the pre-activated catalyst were introduced to a 125 mL Parr reactor with a magnetic stirrer. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. Afterwards, the gases inside the vessel were partly collected in a gas sampling bag for GC analysis whereas the remaining gas was slowly released. The methanol formed can be detected by ¹H NMR and isolated in vacuo. The results of the hydrogenation of the CO₂ capture with KOH to methanol following the method described here are shown in Table 5.

TABLE 5
CO₂ capture and hydrogenation to methanol

| | | | | CO₂ | Methanol | Methanol |
|---|---|---|---|---|---|---|
| | Base | Time | CO₂ | captured | produced | yield |
| Entry | (mmol) | (h) | source | (mmol) | (mmol) | (%) |
| 1 | KOH | 3 | pure CO₂ | 10.0 | 10.0 | >99 |
| 2 | (10) | 72 | indoor air | 6.6 | 6.4 | 97 |

Capture conditions: solvent = 10 mL, rt, stirring at 800 rpm. Hydrogenation conditions: H₂ = 70 bar at rt, catalyst loading = 300 mg, T = 200° C., t = 72 h, CH₃OH yields calculated relative to CO₂ captured. Yield calculations error ±5%.

### Example 6. Hydrogenation of ¹³C Labelled Potassium Carbonate to ¹³C Labelled Methanol in Naturally Abundant Methanol and Water Solvent

Commercial Cu/ZnO/Al₂O₃ catalyst was purchased from Alfa Aesar (63.5 wt % CuO, 24.7 wt % ZnO, 10.1 wt % Al₂O₃, 1.3 wt % MgO fume). The pellets were then crushed and sieved to a size of 250 μm or less. For activating the Cu/ZnO/Al₂O₃ catalyst, the catalyst was subjected to a flow of N₂ (100 mL/min) at 120° C. for 1 h, then ramped up to 270° C. (at 10° C./min) with a H₂ flow (35 mL/min) in N₂ (100 mL/min) at 1 atm and kept at that temperature for 5 h. The so treated catalyst was then stored under Argon for use at a later time. In a nitrogen-filled chamber, the ¹³C labelled potassium carbonate, pre-activated catalyst and the solvents were introduced to a 125 mL Parr reactor with a magnetic stirrer. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. Afterwards, the gases inside the vessel were partly collected in a gas sampling bag for GC analysis whereas the remaining gas was slowly released. The ¹³C methanol formed was detected by 1H NMR and isolated in vacuo. The results of the hydrogenation of potassium carbonate to methanol following the method described here are shown in Table 6.

TABLE 6
Hydrogenation of ¹³C labelled potassium carbonate to ¹³C
labelled methanol in naturally abundant methanol and water solvent

| | ¹³C | | | CH₃OH | CH₃OH |
|---|---|---|---|---|---|
| | substrate | | time | Produced | yield |
| Entry | (mmol) | solvent (mL) | (h) | (mmol) | (%) |
| 1 | K₂CO₃ (5) | methanol (5 mL) | 72 | 3.1 | 62 |
| 2 | K₂CO₃ (5) | methanol (5 mL) + | 72 | 3.9 | 78 |
| | | water (0.5 mL) | | | |

Hydrogenation conditions: H₂ = 80 bar at rt, Cu/ZnO/Al₂O₃ loading = 600 mg, T = 200° C., t = 72 h. Yield calculations error ±5%.

### Example 7. Hydrogenation of Metal Carbonates and Bicarbonates to Methane with Ni/CaAl₂O₄ in Water

Commercial HIFUEL R110 Ni/CaAl₂O₄ catalyst was purchased from Alfa Aesar. For activating the Ni/CaAl₂O₄ catalyst, the catalyst was subjected to a flow of N₂ (100 mL/min) at 120° C. for 1 h, then ramped up to 700° C. (at 10° C./min) with a H₂ flow (35 mL/min) in N₂ (100 mL/min) at 1 atm and kept at that temperature for 6 h. The so treated catalyst was then stored under Argon for use at a later time. In a nitrogen-filled chamber, potassium bicarbonate or carbonate salts, pre-activated catalyst and water were introduced to a 125 mL Parr reactor. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. The methane formed was analyzed by GC. The results of the hydrogenation of potassium carbonate and potassium bicarbonate to methane following the method described here are shown in Table 7.

TABLE 7
Hydrogenation of metal carbonates and bicarbonates
to methane with Ni/CaAl₂O₄ in water

| | | substrate | | | | Methane | |
|---|---|---|---|---|---|---|---|
| | | amount | H₂ | Temperature | Time | produced | Methane |
| Entry | substrate | (mmol) | (bar) | (° C.) | (hours) | (mmol) | yield (%) |
| 1 | KHCO₃ | 10 | 50 | 170 | 48 | 0.6 | 6 |
| 2 | KHCO₃ | 10 | 50 | 200 | 48 | 0.7 | 7 |
| 3 | KHCO₃ | 10 | 50 | 225 | 48 | 9.7 | 97 |
| 4 | KHCO₃ | 10 | 40 | 225 | 48 | 5.4 | 54 |
| 5 | KHCO₃ | 10 | 50 | 225 | 48 | 9.7 | 97 |
| 6 | KHCO₃ | 10 | 60 | 225 | 48 | 8.2 | 82 |
| 7 | KHCO₃ | 10 | 70 | 225 | 48 | 6.3 | 63 |
| 8 | KHCO₃ | 10 | 50 | 225 | 24 | 2.2 | 22 |
| 9 | KHCO₃ | 10 | 50 | 225 | 72 | 9.8 | 98 |
| 10 | K₂CO₃ | 10 | 50 | 225 | 48 | 5.3 | 53 |

Reaction conditions: water (10 mL), H₂ pressure at room temperature, 300 mg Ni/CaAl₂O₄. Methane yields were calculated relative to the carbonate as determined by gas chromatography. Yield calculations ±5%.

### Example 8. Hydrogenation of Metal Carbonates and Bicarbonates to Methane with Ni/Al₂O₃ in Water

The catalyst used in the experiments in this example was composed of 33 wt % Ni on Al₂O₃. For activating the Ni/Al₂O₃ catalyst, the catalyst was subjected to a flow of N₂ (100 mL/min) at 120° C. for 1 h, then ramped up to 700° C. (at 10° C./min) with a H₂ flow (35 mL/min) in N₂ (100 mL/min) at 1 atm and kept at that temperature for 6 h. In a nitrogen-filled chamber, potassium bicarbonate or carbonate salts, pre-activated catalyst and water were introduced to a 125 mL Parr reactor. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes. The methane formed was analyzed by GC. The results of the hydrogenation of some metal carbonates and bicarbonates to methane following the method described here are shown in Table 8.

TABLE 8
Hydrogenation of metal carbonates and bicarbonates
to methane with Ni/Al₂O₃ in water

| | | substrate | | | | Methane | |
|---|---|---|---|---|---|---|---|
| | | amount | H₂ | Temperature | Time | produced | Methane |
| Entry | substrate | (mmol) | (bar) | (° C.) | (hours) | (mmol) | yield (%) |
| 1 | KHCO₃ | 10 | 50 | 225 | 48 | 10 | 100 |
| 2 | K₂CO₃ | 10 | 50 | 225 | 48 | 10 | 100 |
| 3 | K₂CO₃ | 20 | 50 | 225 | 48 | 11 | 55 |
| 4 | Na₂CO₃ | 10 | 50 | 225 | 48 | 10 | 100 |
| 5 | CaCO₃ | 10 | 50 | 225 | 48 | 1 | 10 |

Reaction conditions: water (10 mL), 50 bar H₂ pressure at room temperature, 48 h, 300 mg Ni/Al₂O₃. Methane yields calculated relative to the carbonate as determined by gas chromatography. Yield calculations ±5%.

### Example 9. Hydrogenation of Potassium Carbonate to Methane with Ni/Al₂O₃ in Water, Over Several CO₂ Absorption/Metal Carbonate Reduction Cycles

The catalyst used in the experiments in this example was composed of 33 wt % Ni on Al₂O₃. For activating the Ni/Al₂O₃ catalyst, the catalyst was subjected to a flow of N₂ (100 mL/min) at 120° C. for 1 h, then ramped up to 700° C. (at 10° C./min) with a H₂ flow (35 mL/min) in N₂ (100 mL/min) at 1 atm and kept at that temperature for 6 h. The so treated catalyst was then stored under Argon for use at a later time. Separately, a solution of KOH (4 mmol) in water (10 mL) was prepared in a flask. The flask was then purged to remove any atmosphere and placed under a pure CO₂ atmosphere while the solution was stirred at 800 rpm. The amount of CO₂ captured was measured by both the volume of CO₂ added and gravimetrically. In a nitrogen-filled chamber, the solution resulting from the reaction of KOH with CO₂, and the pre-activated catalyst were introduced to a 125 mL Parr reactor. The sealed vessel was then filled to the desired pressure with H₂. The reactor was placed in a preheated aluminum block and heated to the desired temperature. After heating for a given reaction period, the reactor was cooled to room temperature. The vessel was then cooled in an ice bath for 30 minutes and then pressure was released. Part of the gas released was collected into a collection bag for gas chromatography analysis to determine the amount of methane formed. The liquid was decanted from the catalyst and placed in a 100 mL round bottom flask. The flask was then purged to remove any atmosphere and placed under a pure CO₂ atmosphere while the solution was stirred at 800 rpm. The amount of CO₂ captured was measured by both the volume of CO₂ added and gravimetrically. The obtained solution was then used again in the hydrogenation reaction to methane as described earlier in this paragraph. This operation was repeated four more times after that to check the recyclability of both the catalyst and the base/carbonate species. The results of these consecutive CO₂ capture/hydrogenation reactions to methane following the method described here are shown in Table 9.

TABLE 9
Hydrogenation of potassium bicarbonate to methane
with Ni/Al₂O₃ in water, over several CO₂ absorption/metal
carbonate reduction cycles.

| | | | Amount | | |
|---|---|---|---|---|---|
| | | | of CO₂ | Methane | Methane |
| | Temperature | Pressure | captured | produced | Yield |
| Cycle | (° C.) | H₂ (bar) | (mmol) | (mmol) | (%) |
| 1 | 225 | 50 | 4 | 4 | 100 |
| 2 | 225 | 50 | 4 | 4 | 100 |
| 3 | 225 | 50 | 4 | 4 | 100 |
| 4 | 225 | 50 | 4 | 3.95 | 100 |
| 5 | 225 | 50 | 3.95 | 3.95 | 100 |

Capture conditions: KOH (4 mmol), water (10 mL), t = 48 h, stirring at 800 rpm, rt, Methanation conditions: water (10 mL), 50 bar H₂ pressure at room temperature, 225° C., 300 mg Ni/Al₂O₃. Methane yields calculated relative to the carbonate as determined by Gas chromatography. Yield calculations ±5%.

### Example 10. Hydrogenation of Potassium Carbonate to Methane Over Various Catalysts in Water

TABLE 10
Hydrogenation of potassium carbonate to
methane with various catalysts in water.

| | | Methane | Methane |
|---|---|---|---|
| | | produced | yield |
| Entry | Catalyst | (mmol) | (%) |
| 1 | 33% Ni/Al₂O₃ | 6.4 | 64 |
| 2 | 5% Ru/Al₂O₃ | 10 | 100 |
| 3 | 5% Rh/Al₂O₃ | 1.4^{[a]} | 14^{[a]} |
| 4 | 12% Ni/Al₂O₃ | 1.2 | 12 |
| 5 | 12% Ni/3% Y/Al₂O₃ | 6.9 | 69 |
| 6 | 12% Ni/3% La/Al₂O₃ | 6.2 | 62 |
| 7 | 12% Ni/3% Ce/Al₂O₃ | 4.8 | 48 |
| 8 | 12% Ni/3% Pr/Al₂O₃ | 6.3 | 63 |
| 9 | 12% Ni/3% Nd/Al₂O₃ | 6.3 | 63 |
| 10 | 12% Ni/3% Sm/Al₂O₃ | 6.4 | 64 |
| 11 | 12% Ni/3% Gd/Al₂O₃ | 6.9 | 69 |
| 12 | 12% Ni/3% Dy/Al₂O₃ | 6.9 | 69 |
| 13 | 12% Ni/3% Yb/Al₂O₃ | 6.9 | 69 |

Reaction conditions: water (10 mL), 10 mmol K₂CO₃, 50 bar H₂ pressure at room temperature, reaction temperature 225° C., 24 h, 300 mg catalyst. Methane yields calculated relative to the carbonate as determined by gas chromatography. Yield calculations ±5%.
^{[a]}Also produced 2.4 mmol of CO₂.

### Example 11. Hydrogenation of Metal Carbonates to Methanol Using Ni₅Ga₃/Gd/SiO₂

TABLE 11
Hydrogenation of metal carbonates
to methanol using Ni₅Ga₃/Gd/SiO₂.

| | | | Products | | |
|---|---|---|---|---|---|
| | | Metal | Formate | Methanol | Methanol |
| Trial | Catalyst | carbonate | (mmol) | (mmol) | Yield (%) |
| 1 | Ni₅Ga₃/Gd/SiO₂ | K₂CO₃ | 1.49 | 6.03 | 60 |
| 2 | Ni₅Ga₃/Gd/SiO₂ | Na₂CO₃ | Traces | 6.75 | 68 |
| 3 | Ni₅Ga₃/Gd/SiO₂ | Rb₂CO₃ | 4.49 | 1.82 | 18 |
| 4 | Ni₅Ga₃/Gd/SiO₂ | Li₂CO₃ | traces | 7.79 | 78 |

Reaction conditions: ethylene glycol (5 mL), 10 mmol metal carbonate, 60 bar H₂ pressure at room temperature, reaction temperature 225° C., 48 h, 300 mg catalyst. CH₃OH yields calculated relative to the carbonate as determined by ¹H NMR. Yield calculations ±5%.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

[1] Keith, D. W.; Holmes, G.; St. Angelo, D.; Heidel, K., A Process for Capturing CO₂ from the Atmosphere. Joule 2018, 2 (8), 1573-1594.
[2] Goeppert, A.; Czaun, M.; Prakash, G. K. S.; Olah, G. A., Air as the renewable carbon source of the future: an overview of CO₂ capture from the atmosphere. Energ. Environ. Sci 2012, 5 (7), 7833-7853.
[3] Goeppert, A.; Czaun, M.; Jones, J.-P.; Prakash, G. K. S.; Olah, G. A., Recycling of carbon dioxide to methanol and derived products-closing the loop. Chem. Soc. Rev. 2014, 43, 7995-8048.
[4] McQueen, N.; Kelemen, P.; Dipple, G.; Renforth, P.; Wilcox, J., Ambient weathering of magnesium oxide for CO₂ removal from air. Nature Communications 2020, 11 (1), 3299.

What is claimed is:

1. A method for conversion of a carbonate/formate component comprising:
contacting the carbonate/formate component with hydrogen to produce methanol, methane, carbon monoxide or hydrocarbons or a mixture thereof over a catalyst in a solvent, the catalyst including a transition metal, a post-transition metal, a lanthanide, or combinations thereof.

2. The method of claim 1, wherein the catalyst is heterogeneous.

3. The method of claim 1, wherein the carbonate/formate component is a metal carbonate, metal bicarbonate, metal formate salt, or mixtures thereof.

4. The method of claim 1, wherein a metal hydroxide is produced as a co-product.

5. The method of claim 1, wherein the carbonate/formate component is a metal carbonate selected from the group consisting of lithium carbonate, sodium carbonate, potassium carbonate, rubidium carbonate, cesium carbonate, magnesium carbonate, calcium carbonate, strontium carbonate, barium carbonate, and mixtures thereof.

6. The method of claim 1, wherein the carbonate/formate component is a metal bicarbonate selected from the group consisting of lithium bicarbonate, sodium bicarbonate, potassium bicarbonate, rubidium bicarbonate, cesium bicarbonate, and mixtures thereof.

7. The method of claim 1, wherein the carbonate/formate component is a metal formate salt selected from the group consisting of lithium formate, sodium formate, potassium formate, rubidium formate, cesium formate, and mixtures thereof.

8. The method of claim 1, wherein the solvent comprises water, an alcohol, a diol, a polyol or a mixture thereof.

9. The method of claim 1, wherein the solvent comprises methanol, ethanol, isopropanol, n-butanol, ethanol propanol, 2-methoxyethanol, ethylene glycol, diethylene glycol, triethylene glycol, tetraethylene glycol, polyethylene glycol, propylene glycol, 1,4-butanediol or a mixture thereof.

10. The method of claim 1, wherein the carbonate/formate component is a metal carbonate and/or a metal bicarbonate each independently including a component selected from the group consisting of:
naturally occurring minerals;
seashells, oyster shells, eggshells;
carbonates and bicarbonate salts made by direct CO₂ capture from CO₂ sources using alkali hydroxides and/or amines;
synthetic carbonates and bicarbonates; and
combinations thereof.

11. The method of claim 1, wherein the carbonate/formate component includes naturally occurring minerals are selected from the group consisting of calcite (CaCO₃), magnesite (MgCO₃), siderite (FeCO₃), aragonite (CaCO₃), witherite (BaCO₃), natrite (Na₂CO₃), ankerite (CaFe—CO₃), dolomite (CaMg(CO₃)₂), huntite (Mg₃Ca(CO₃)₄), minrecordite (CaZn(CO₃)₂), barytocalcite (BaCa(CO₃)₂), hydromagnesite (Mg₅(CO₃)₄(OH)₂·4H₂O), ikaite (CaCO₃·6(H₂O)), lansfordite (MgCO₃·5(H₂O)), monohydrocalcite (CaCO₃·H₂O), natron (Na₂CO₃·10(H₂O)), and combinations thereof.

12. The method of claim 1, wherein the hydrogen is produced from a fossil or renewable source.

13. The method of claim 1, wherein a heterogeneous catalyst system includes the catalyst.

14. The method of claim 13, wherein the heterogeneous catalyst system includes a component selected from the group consisting of copper-based catalysts, indium-based catalysts, nickel-based, indium and nickel/gallium-based catalysts modified with lanthanides and/or precious metals, and combinations thereof.

15. The method of claim 13, wherein the heterogeneous catalyst system further includes additional components selected from the group consisting of additives, supports, and combinations thereof.

16. The method of claim 15, wherein the additional components are selected from the group consisting of ZnO, ZrO₂, MOFs, Ga, Al₂O₃, SiO₂. TiO₂, and combinations thereof.

17. The method of claim 13, wherein the heterogeneous catalyst system includes an indium-based species having the formula In₂O₃/ZrO₂.

18. The method of claim 13, wherein the heterogeneous catalyst system includes a nickel-based species selected from the group consisting of NiGa/SiO₂, Ni₃Ga/SiO₂, and Ni₅Ga₃/SiO₂.

19. The method of claim 1, wherein the catalyst comprises a heterogeneous catalyst based on nickel, cobalt, ruthenium, platinum, palladium, iron, iridium or rhodium and combinations thereof either in their pure form or deposited on a support.

20. The method of claim 19, wherein the support is selected from the group consisting of silica, alumina, zirconia, titanium oxide, cerium oxide, silica-alumina, and mixtures thereof.

21. The method of claim 1, wherein the catalyst includes a heterogeneous catalyst selected from the group consisting of Fischer-Tropsch type catalysts based on iron, cobalt, and/or ruthenium and catalysts based on iron, copper, molybdenum, cobalt, metal carbides, zeolites or mixtures thereof.

22. The method of claim 1, wherein the conversion is conducted at temperatures between 50° C. and 600° C. and gauge pressures between 0 and 200 bar.

23. The method of claim 1, wherein the carbonate/formate component is a metal carbonate and/or a metal bicarbonate which is the product of a reaction of a metal hydroxide with CO₂ contained in CO₂ sources.

24. The method of claim 23, wherein the CO₂ sources include a CO₂ source selected from the group consisting of fossil fuel power plant flue gases, emissions from industrial and commercial sources, and CO₂ contained in air.

25. The method of claim 23, wherein the metal hydroxide comprises lithium hydroxide, sodium hydroxide, potassium hydroxide, rubidium hydroxide, cesium hydroxide, magnesium hydroxide, calcium hydroxide, strontium hydroxide, barium hydroxide or mixtures thereof.

26. The method of claim 1 wherein the carbonate/formate component reacts to form a metal hydroxide or a metal hydroxide mixture.

27. The method of claim 26, wherein the metal hydroxide or the metal hydroxide mixture is used to capture CO₂ from sources containing between 0.04% and 100% CO₂, including ambient air, indoor air, industrial flue gases, fossil fuel burning power plants, natural gas purification facilities, cement plants, breweries, biogas, biomass burning facilities and biomass gasification facilities to obtain a metal carbonate, metal bicarbonate or a mixture thereof.

28. The method of claim 1, wherein the carbonate/formate component includes a metal carbonate, metal bicarbonate or mixtures thereof, the carbonate/formate component reacting to form a metal hydroxide or metal hydroxide mixture and wherein the metal hydroxide or the metal hydroxide mixture obtained therefrom is used to capture CO₂ from sources containing between 0.04% and 100% CO₂ to obtain the metal carbonate, metal bicarbonate or mixtures thereof and wherein the metal carbonates, metal bicarbonates or mixtures thereof are reusable.

29. The method of claim 28, wherein the sources are selected from the group consisting of ambient air, indoor air, industrial flue gases, fossil fuel burning power plants, natural gas purification facilities, cement plants, breweries, biogas, biomass burning facilities and biomass gasification facilities.

30. The method of claim 1 run either under batch conditions or continuous conditions.

31. A method for producing methanol, methane, carbon monoxide, hydrocarbons, or a mixture thereof from CO₂ comprising the following steps:
a) capturing CO₂ with a metal hydroxide or metal hydroxide mixture from a CO₂ source that includes between 0.04% and 100% CO₂ to obtain metal carbonates, metal bicarbonates or mixtures thereof;
b) converting the metal carbonates, metal bicarbonates and mixtures thereof with hydrogen to produce methanol, methane, carbon monoxide, hydrocarbons, or a mixture thereof over a catalyst in a solvent while regenerating the metal hydroxide or the metal hydroxide mixture; and
c) reusing the metal hydroxide or the metal hydroxide mixture is to capture CO₂ as described in step (a).

32. The method of claim 31, wherein the CO₂ source is selected from the group consisting of ambient air, indoor air, industrial flue gases, fossil fuel burning power plants, natural gas purification facilities, cement plants, breweries, biogas, biomass burning facilities, and biomass gasification facilities
